(19) United States
(12) Patent Application Publication
(10) Pub. No.: US 2011/0262565 A1
(43) Pub. Date: Oct. 27, 2011

(54) PIP-2 Inhibition-Based Antiviral and Anti-Hyperlipidemic Therapies

(76) Inventors: Jeffrey S. Glenn, Palo Alto, CA (US); Phillip S. Pang, Palo Alto, CA (US); Nam-Joon Cho, Stanford, CA (US); Choongho Lee, Palo Alto, CA (US)

(21) Appl. No.: 12/994,414

(22) PCT Filed: May 29, 2009
(86) PCT No.: PCT/US09/03271

§ 371 Date: Jul. 6, 2011

Related U.S. Application Data
(60) Provisional application No. 61/057,188, filed on May 29, 2008.

(51) Int. Cl. A61K 33/00; A61K 31/7036; A61P 31/12; G01N 33/53

(52) U.S. Cl. 424/722; 436/501; 514/39

(57) ABSTRACT

Interaction of a specific viral domain with phosphatidylinositol 4,5-bisphosphate (PIP 2) is shown to mediate viral replication. Basic Amino Acid PIP-2 Pincer (BAAPP) domains are described herein, including, without limitation, NS5A protein of HCV, NS4B protein of HCV, poliovirus, and rhinovirus.

### BACKGROUND OF THE INVENTION

Hepatitis C Virus (HCV) is a global health problem with estimates of more than 2% of the world's population currently infected with the virus. One of the outstanding characteristics of HCV is its ability to establish chronic infections in 65-80% of infected patients. Chronic infection with HCV can lead to serious sequelae including chronic active hepatitis, cirrhosis and hepatocellular carcinoma—usually manifested 10, 20 and 25 years respectively after the initial infection. End stage liver disease from HCV has become the leading indication for liver transplantation in North America, and it has been suggested that there will be a 2-3 fold increase in liver transplantation in 10 years as a result of cirrhosis from hepatitis C.

Discovered in 1989, the virus, classified as a Flavivirus, has a 9.5 kilobase positive-strand RNA genome which encodes a single polypeptide of 3008-3037 amino acids long. Based on the genetic variability of the virus, which can be up to 30% at the nucleotide level, at least 6 genotypes and more than 30 subtypes have been identified. This variability has implications for vaccine and antiviral drug development. At present the only approved therapies are interferon, with or without ribavirin, which is not successful in many patients. There is therefore an urgent need to develop novel antivirals to treat HCV.

Many components of the HCV polyprotein and genome have been identified and characterized. The open reading frame (ORF) of HCV is flanked by a non-translated region at the 5′ end, and approximately 200 nucleotides at the 3′ end containing a poly-U tract and a highly conserved 98 base sequence. The core protein located at the N-terminal end of the ORF is the viral capsid protein. The core protein is released from the viral polypeptide by host proteases. In addition to binding to viral RNA, the core protein has also been shown to suppress apoptotic cell death.

The HCV polyprotein is cleaved by a mixture of host and viral proteases. The NS2 gene encodes a zinc²⁺ metalloproteases that produces a cis-cleavage between NS2 and NS3. The cleavage of the NS2/3 junction releases the N-terminus of NS3, the serine protease responsible for the majority of the viral polypeptide cleavages. The first cleavage that the NS3 protease performs liberates the NS4A protein. NS4A contains a highly conserved central domain that is responsible for the efficient NS3 function. The N-terminus encodes a 20 amino acid region that is believed to form a transmembrane domain which thereby anchors NS3 to the endoplasmic reticulum membrane.

Like other positive strand RNA viruses, HCV is believed to replicate in association with cytoplasmic membranes. In the case of HCV, the structures are termed the membranous web and are believed to be induced by the NS4B protein. NS4B is also required to assemble the other viral NS proteins within the apparent sites of RNA replication. The site of viral replication and assembly appears to intersect with host cell pathways of lipid trafficking and lipoprotein production. Amphipathic helices (AHs) have been identified in several HCV NS proteins that mediate membrane association and HCV replication.

The NS5A protein of HCV is a membrane anchored phosphoprotein that is composed of three domains plus an N-terminal amphipathic helix. It precise role has not been determined, but it has been shown to play a role in RNA binding, multiple host-protein interactions, and inteferon resistance. Its N-terminal amphipathic helix has been show to be critical for viral replication and membrane anchoring.

There is an ongoing need in the art for agents that treat HCV infection; and for methods of identifying candidate agents that are suitable for treating HCV infection.

### SUMMARY OF THE INVENTION

Compositions and methods are provided for the treatment of viral infections. A novel approach is provided for the treatment of a broad range of viruses, based on the discovery that interaction of a specific viral domain with phosphatidylinositol 4,5-bisphosphate (PIP-2) mediates viral replication. In some embodiments of the invention, the virus is hepatitis C virus (HCV). In some embodiments of the invention, the viral infection is treated by contacting a patient with a biologically active agent that interferes with the interaction between PIP-2 and an amphipathic helix domain of the virus. Representative Basic Amino Acid PIP-2 Pincer (BAAPP) domains contained in a variety of proteins' amphipathic helices are described herein, including, without limitation, NS5A protein of HCV, NS4B protein of HCV, and proteins of poliovirus, and rhinovirus. In some embodiments, the biologically active agent is a ligand of PIP-2. In other embodiments, the biologically active agent is an analog of PIP-2 that binds to the BAAPP. In other embodiments the biologically active agent with interaction between the BAAPP domain and PIP-2. In other embodiments the biologically active agent interferes with interferes with the proper folding of the BAAPP domain, or the enzymatic pathways responsible for production of PIP-2 or stimulate the phosphatase responsible for its destruction. Biologically active agents of interest include, without limitation, neomycin and derivatives thereof, and lithium. The agents may be formulated or provided in combination with a second antiviral agent, e.g. interferon, ribivarin, and the like. For treatment of viruses such as HCV, the agents may be formulated to specifically target the liver, e.g. by conjugation with polyarginine or a bile acid, or as pro-drugs designed to be activated by enzymes resident in the liver.

Evidence is provided herein that the N-terminal amphipathic helix (AH) of HCV nonstructural protein 5A (NS5A) specifically binds lipid vesicles containing PIP-2. PIP-2 binding induces a significant conformational change in the AH. Cytosolic PIP-2 domains can help identify the sites of active HCV replication. A pair of highly conserved positively-charged lysine residues within the AH were found to be critical for mediating both PIP-2 binding and RNA genome replication. Such positively charged residues project from the hydrophobic side of the AH so as to form a so-called Basic Amino Acid PIP-2 Pincer (BAAPP) domain that is conserved in the NS5A AH across all HCV isolates as well as the AHs found in other viral proteins and host cell apolipoproteins.

In some embodiments, methods are provided for screening drug candidates for antiviral activity, by determining the effect of the agent on the interaction between a viral BAAPP domain and PIP-2, including high throughput assays. Such assays may include detection of the PIP2:BAPP domain interaction by fluorescence polarization. In other embodiments, methods are provided for determining the presence of a BAAPP domain in a protein.

In other embodiments, it is demonstrated that certain lipoproteins contain a BAAPP domain, where biologically active agents that interfere with the interaction between PIP-2 and an amphipathic helix domain of the lipoprotein are useful in inhibiting the formation of LDL or VLDL particles. In some embodiments, the biologically active agent is a ligand of PIP-2. In other embodiments, the biologically active agent is an analog of PIP-2 that binds to the BAAPP. In other embodiments the biologically active agent interferes with the interaction between the BAAPP domain and PIP-2. In other embodiments the biologically active agent interferes with interferes with the proper folding of the BAAPP domain, or the enzymatic pathways responsible for production of PIP-2 or stimulate the phosphatase responsible for its destruction. The agents may be formulated or provided in combination with a second therapeutic agent.

These and other advantages, and features of the invention will become apparent to those persons skilled in the art upon reading the details of the compositions and methods of use are more fully described below.

### BRIEF DESCRIPTION OF THE DRAWINGS

FIG. 1. The NS5A amphipathic helix specifically binds lipid vesicles containing PIP-2. QCM-D measurements, quantifying mass changes due to the binding of the NS5A amphipathic helix to various PIPs. The system consists of polymerized vesicles containing the indicated PIPs deposited intact on a SiO2 solid substrate. We employed the Sauerbrey equation to covert frequency to a real mass of bound peptide. AH peptides significantly bind on PIP45, not PIP, PIP34, and PIP35. As a positive control, we utilized GRIP, which is a recombinant PLC-d1 PH domain GST-tagged protein (2.5 ug lyophilized) that is known to bind PI(4,5)P2 (PIP2). As a negative control, we employed the NH peptide, in which three point mutations were introduced into the AH to disrupt amino acids on the hydrophobic face. Notably, the NH peptide shows no significant binding to any of the target lipid vesicles. Abbreviations: AH: amphipathtic helix; NH: Mutant Amphipathic Helix (Journal of Virology, May 2003, p. 6055-6061, Vol. 77, No. 10); PIP: phosphatidylinositol; PIP34: phosphatidylinositol 3,4-bisphosphate; PIP35: phosphatidylinositol 3,5-bisphosphate; PIP45: phosphatidylinositol 4,5-bisphosphate, also called PIP-2; GRIP: Positive Control Peptide.

FIG. 2. PIP-2 binding is mediated by a pair of conserved positively-charged amino acids. Molecular surface model of the BAAPP domain of NS5A created using the program DeepView/Swiss-PdbViewer. The surface is colored by electrostatic potential, using a gradient from red to blue, with blue denoting positive electrostatic potentials, white denoting neutral potentials, and red denoting negative electrostatic potentials. In right panel, helix wheel plot of NS5A BAAPP domain, with hydrophobic face denoted by the yellow pie slice and filled yellow circles. Positively charged residues that flank the hydrophobic face are indicated by the filled-in blue circles, with blue denoting that they are positively charged residues. On left, molecular surface model of the BAAPP domain of NS5A created using the program DeepView/Swiss-PdbViewer. The surface is colored by electrostatic potential, using a gradient from red to blue, with blue denoting positive electrostatic potentials, white denoting neutral potentials, and red denoting negative electrostatic potentials. Mutations (as indicated, K20A, K26A, and K20AK26A) in the molecular model were made using DeepView. Helix wheel plots of mutant NS5A BAAPP domains, with hydrophobic face denoted by the yellow pie slice and filled yellow circles. On right, far-UV circular dichroism (CD) analyses of AH peptide (from NS5A) and the single mutants K20A and K26A and the double-mutant K2OAK26A. The CD spectra were recorded in 10 mM PBS buffer, pH 7.5. QCM-D measurements of the binding of the AH peptide and mutant variants thereof to PIP45 containing vesicles, using the same technique as in FIG. 1. QCM-D kinetic adsorption data of Δf n=overtones/n versus time for 0.025 mg/ml vesicles in PBS buffer solution (250 mM NaCl, pH 7.5) is shown in top left panel. Vesicles were added after stabilizing the frequency signal for 10 min. The film was then washed twice with buffer and then the indicated AH peptide were added. NOTE: only the AH peptide bound to the vesicle platform, its mutant variants did not. Two buffer washes were performed in order to ensure the stability of the film. Top Right Panel: Corresponding dissipation changes as a function of time is shown in top right panel. Bottom Left: we employed the Sauerbrey equation to covert frequency to the real mass of bound peptide. Abbreviations: K20A: Lysine 20 to Alanine Mutation; K26A: Lysine 26 to Alaine Mutation; K20AK26A: double mutant consisiting of both K20A and K26A; PIP2: phosphatidylinositol 4,5-bisphosphate, also called PIP45. AH: amphipathtic helix; NH: Not-Amphipathic Helix (Journal of Virology, May 2003, p. 6055-6061, Vol. 77, No. 10); NH20: same as K20A, which is the AH with Lysine 20 to Alanine Mutation; NH26, same as K26A, which is the Lysine 26 to Alaine Mutation; NH20,26: same as K20AK26A, which is the double mutant consisiting of both K20A and K26A.

FIG. 3. NS5A co-localizes with PIP-2 only in the context of the HCV replication complex. Huh7 cells were transfected with expression vectors for wild type NS5A or mutant NS5A (K20AK26A) proteins fused in frame to the N-terminus of eGFP. At 24 hr after transfection, PIP-2 was visualized (in red) by immunofluorescence using a monoclonal anti-PIP-2 antibody. Huh7 cells were cotransfected with expression vectors for wild type NS5A-GFP and mutant NS5A (K20AK26A)-DsRed proteins. Subcellular localization of PIP-2 was examined in RPY21 cells harboring replicating HCV replicons with an YFP-tagged version of NS5A (top panels) and in RP7 cells harboring Bart79I replicating HCV subgenomic replicons, wherein NS5A was visualized by immunofluorescence using a monoclonal anti-NS5A antibody (bottom panels).

FIG. 4. PIP-2 binding induces a conformation change in the NS5A amphipathic helix. 2C Far-UV circular dichroism (CD) analyses of AH peptide derived from NS5A in membrane mimetic environments. The CD spectra were recorded in 10 mM PBS buffer, pH 7.5 for AH peptide alone, or AH peptide plus 120 mM of PIP34- or PIP45-containing polymerized vesicles. PIP34:phosphatidylinositol 3,4-bisphosphate; PIP45: phosphatidylinositol 4,5-bisphosphate, also called PIP-2.

FIG. 5. PIP-2 mediates HCV RNA genome replication. Colony formation assay. Huh 7.5 cells were electroporated with 5ug of in vitro-transcribed wild type Bart79I, mutant Bart79I encoding NS5A (K20AK26A), or Bart79I (Pol-) RNAs followed by selection with 750 ug/ml of G418 for three weeks. Surviving colonies were stained with crystal violet and the number of colonies was counted from three different plates to calculate average number of colonies and standard deviation. Reversion to wild-type sequence. Left panel shows the sequence of input mutant (K20AK26A) HCV replicon RNA. Sequence analysis of replicon RNA isolated from colonies growing on mutant plate from colony formation assay is shown in right panel. Luciferase reporter-linked transient HCV replication assays. Huh 7.5 cells were electroporated with 10ug of in vitro-transcribed wild type Bart79I-luc, mutant Bart791-luc (K20AK26A), or Bart79I-luc (Pol-) RNAs. Firefly luciferase activities were measured at 8, 48, 96, and 144 hours post electroporation.

FIG. 6. A Basic Amino Acid PIP-2 Pincer (BAAPP) domain is found in the amphipathic helices of other important proteins. (6A): Helix Wheel models of various BAAPP domains, first generated using EMBOSS Pepwheel, then further illustrated using powerpoint graphics. Hydrophobic face denoted by the yellow pie slice and filled yellow circles. Positively charged residues that flank the hydrophobic face are indicated by the filled-in blue circles, with blue denoting that they are positively charged residues. Abbreviations: HCV: Hepatitis C Virus; NS5A: Non-Structural Protein 5A; NS4B AH1: Non-Structural Protein 4B, Amphipathic Helix 1; JEV: Japanese Encephalitis Virus; Apo: Apolipoprotein. (6B) and (6C): Helix wheel models, direct output from EMBOSS pepwheel program of various BAAPP domains from various pathogens. 2C: Viral Protein 2C; Apo: Apolipoprotein.

FIG. 7. A PIP-2 ligand inhibits HCV replication in a dose-dependent manner. Luciferase reporter-linked HCV replication assay. Huh 7.5 cells were electroporated with 10 ug of in vitro-transcribed wild type, FL-J6/JFH-5′C19Rluc2AUbi RNAs. Electroporated cells were treated with 0, 172, 345, 689, 1378, and 2756 μM of neomyin. Renilla luciferase activities were measured at 5 days after electroporation. Alamar blue assays were performed to compare relative cell viabilities.

FIG. 8. The 4-5 phosphoinositide (PIP-2)-binding BAAPP domain in HCV NS5A that is essential for viral genome replication is found in other important pathogens. (A) Space-filling model of the HCV NS5A amphipathic helix with the pair of basic lysine residues (K20 and K26) essential for PIP2 binding colored in blue. (B) Helix wheel diagram of HCV NS5A amphipathic helix. Similar BAAPP domains located in (C) P. falciparum PfNDH2 protein essential for parasite mitochondrial function, (D) Japanese encephalitis virus core (E) Dengue virus core proteins that are essential for virus assembly.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a peptide” includes a plurality of such peptides and reference to “the inhibitor” includes reference to one or more inhibitors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The Basic Amino Acid PIP2 Pincer (BAAPP) domain, as described herein, provides a mechanism by which a protein or peptide recognizes (including but not limited to binding as well as activation or suppression of activity) PIP-2 (phosphatidylinositol 4,5-bisphosphate [Ptdlns(4,5)P2]). Alterations or variations of the BAAPP domain may result in recognition of other phosphatidylinositol variants.

BAAPP domains are identified herein in multiple organisms, including but not limited to pathogens such as viruses, bacteria, fungi and parasites, as well as hosts, such as the human. BAAPP domain peptides, molecules that mimic the BAAPP domain, enzymes involved in PIP-2 metabolism, and molecules that inhibit or activate the BAAPP domain act in treating infectious diseases as well as affecting host physiology or pathophysiology.

The BAAPP domain is a structure, usually a polymer, and usually a polymer of amino acids, which takes on a conformation that is usually an alpha helix, in which: (a) positive charges, usually in the form of basic amino acids, are positioned such that they help mediate binding to a negatively charged phospholipid(s), such as PIP2; and (b) it is also usually the case that the structure formed has a hydrophobic surface or region that is attracted to or binds to other hydrophobic surfaces, regions or molecules. And example of such a structure is the hydrophobic side of an amphipathic helix. Since PIP2 can be found as part of a lipid membrane, the hydrophobic property of a BAAPP domain favors lipid/membrane attraction, while the positive charges on the BAAPP domain mediate binding to PIP2. As such, the positive charges are usually but need not necessarily be oriented in the same general direction as the hydrophobic surface or region of a BAAPP domain.

The BAAPP domain differs from other PIP2 binding domains that have been previously described (McLaughlin et al. Annu. Rev. Biophys. Biomol. Struct 2002). Other PIP2 binding domains usually are composed of pockets or clefts formed by multiple parts of a protein into which PIP2 fits, or are either unstructured basic peptides or peptides of undetermined structure.

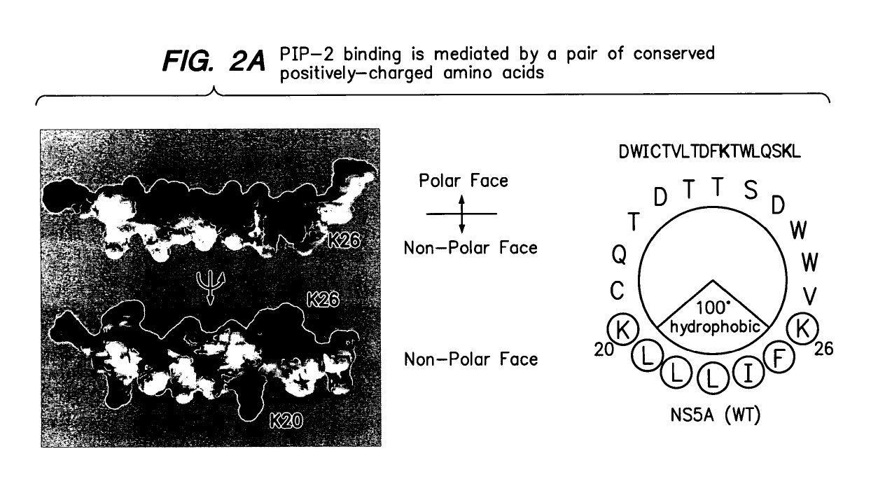

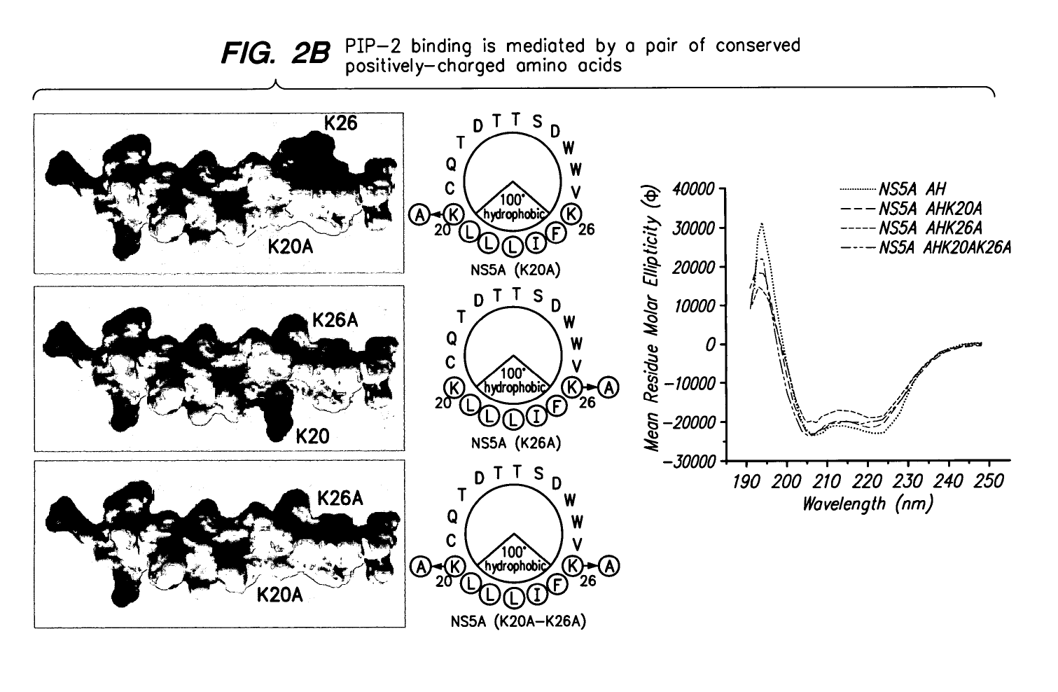

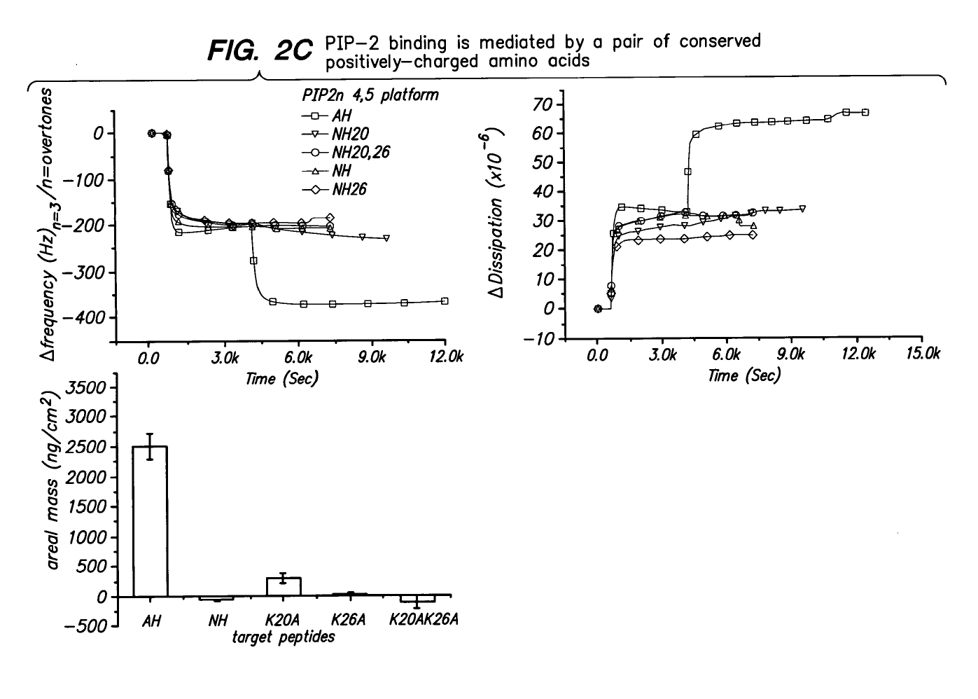

One method of identifying BAAPP domains is to first identify amino acid alpha helices. This identification can be by examination of structural data, such as crystal structure data, or by use of secondary structure prediction analysis of primary sequence data, or some combination of both. Next, a “helix wheel” program can be used to plot or visualize the alpha helix (see FIG. 2). In such helix wheel plots, adjacent amino acids are plotted around a circle, with a 100 degree angle between them. Any method or program that allows for the relative orientation of the amino acid side chains in the helix, with respect to one another, to be determined can be used. Next, such plots can be analyzed, such as by inspection or other means, to determine if the helix under examination has the following properties: (a) a hydrophobic face or surface (b) positive charges, usually in the form of the basic amino acids lysine (K), arginine (R), or histidine (H), that usually flank the hydrophobic face and are oriented in the same general direction as the hydrophobic face.

Mathematical/automated algorithmic processes of identifying amphipathic helices have been described, such as Amphipaseek (Sapay et al. BMC Bioinformatics 2006) or WHEEL, HELNET, COMBO, COMNET, CONSESUS (Jones et al. J of Lipid Research 1992). These methods sometimes use mathematical/algorithmic methods of identifying polypetides that, if helical, would possess hydrophobic faces or surfaces, such as using the method called the hydrophobic moment (Eisenberg et al. PNAS 1984). These methods also sometimes use mathematical/algorithmic methods of secondary structure prediction.

These programs identify regions of a polypeptide(s) that form potential or actual alpha helices with potential or actual hydrophobic faces or regions. They may even result in “helix wheel” plots or other structural plots. They may even result in the identification of what are known as Class A amphipathic helices (Segrest et al. Proteins 1990), which are amphipathic helices with positive charged residues at the hydrophobic-hydrophilic interface and negatively charged residues on the hydrophilic face. Consequently, sequence corresponding to the regions identified by these programs can then be input into a helix wheel program or any other structure plotting program, to determine if the helix under examination has the following properties: (a) a hydrophobic face or surface (b) positive charges, usually in the form of the basic amino acids lysine (K), arginine (R), or histidine (H), that usually flank the hydrophobic face and are oriented in the same general direction as the hydrophobic face, thus identifying a BAAPP domain.

Examples of proteins having a BAAPP domain include, without limitation, the 2C protein of Picornaviridae, Rhinovirus B, Rhinovirus C, PolioVirus, Enterovirus A, Enterovirus B, Enterovirus C, and Enterovirus D. The core protein of Japanese Encephalitis Virus, West Nile Virus, Dengue Virus 1, Dengue Virus 2, Dengue Virus 3, Dengue Virus 4 have BAAPP domains, as does the P. falciparum PfNDH2 protein. In the Flaviviridae, the NS4B AH 1 of HCV; the NS5A protein of HCV which has a BAAPP domain that comprises the conserved lysine residues at residue 20 and 26 of the processed protein, for example a peptide within the amino acid sequence DWICTVLTDFKTVVLQSKL that includes the lysine residues. In some aspects of the invention, analog peptides are provided in which one or both of the lysine residues are substituted, e.g. substituted with alanine, glycine, etc., which peptides lack the PIP-2 binding activity, but have use as negative controls in assays.

BAAPP domains in human proteins are found in Apolipoprotein A, Apolipoprotein C, Apolipoprotein E, Apolipoprotein B; and Gelsolin.

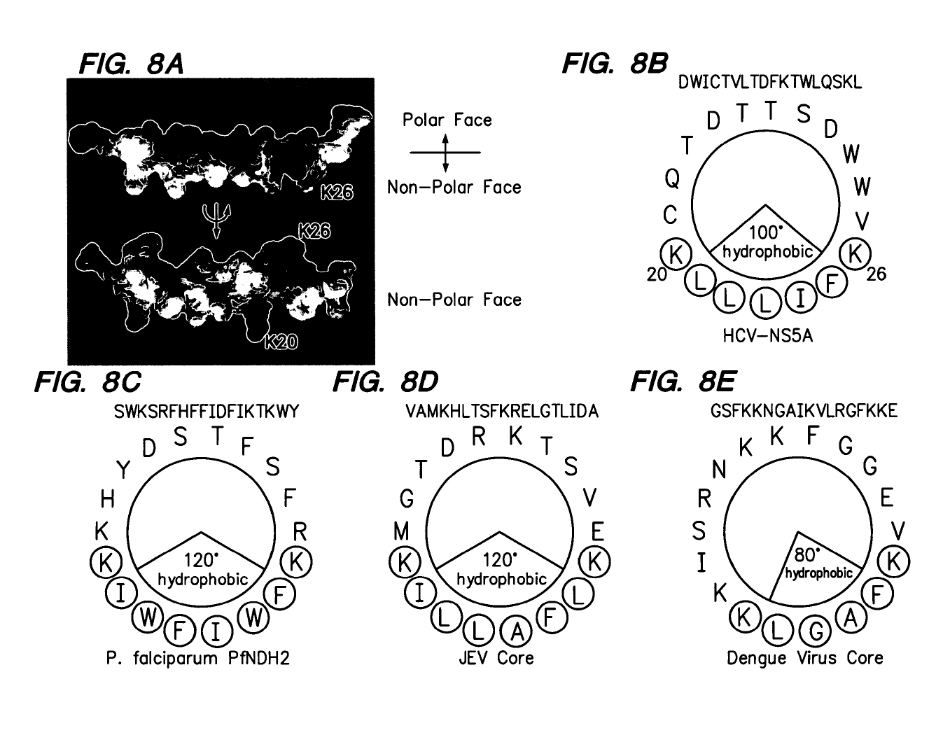

Certain specific domains are shown in FIG. 8. Peptides of interest for assays include, without limitation, a peptide of at least 8 amino acids, at least 10 amino acids, at least 12 amino acids, at least 14 amino acids, and having the conserved binding residues.

NS5 encoding viruses include without limitation flaviviruses, pestiviruses and hepatitis C viruses, e.g. yellow fever virus (YFV); Dengue virus, including Dengue types 1-4; Japanese Encephalitis virus; Murray Valley Encephalitis virus; St. Louis Encephalitis virus; West Nile virus; tick-borne encephalitis virus; Hepatitis C virus; Kunjin virus; Central European encephalitis virus; Russian spring-summer encephalitis virus; Powassan virus; Kyasanur Forest disease virus; and Omsk hemorrhagic fever virus.

By “Flaviviridae virus” is meant any virus of the Flaviviridae family, including those viruses that infect humans and non-human animals. The polynucleotide and polypeptides sequences encoding these viruses are well known in the art, and may be found at NCBI's GenBank database, e.g., as Genbank Accession numbers NC_{—}004102, AB031663, D11355, D11168, AJ238800, NC_{—}001809, NC_{—}001437, NC_{—}004355 NC_{—}004119, NC_{—}003996, NC_{—}003690, NC_{—}003687, NC_{—}003675, NC_{—}003676, NC_{—}003218, NC_{—}001563, NC_{—}000943, NC_{—}003679, NC_{—}003678, NC_{—}003677, NC_{—}002657, NC_{—} 002032, and NC_{—} 001461, the contents of which database entries are incorporated by references herein in their entirety.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified. For example, an isolated peptide of the invention is at least 60%, by weight, free from the proteins and naturally-occurring organic molecules with which it is naturally associated or, in the context of synthetic peptides, at least 60% by weight free of synthetic peptides of different sequence and intermediates. Preferably, the preparation is at least 75%, more preferably at least 90%, and most preferably at least 99%, by weight, peptide. An isolated peptide as described herein may be obtained, for example, by chemically synthesizing the protein or peptide, or by expression of a recombinant nucleic acid encoding a peptide of interest, with chemical synthesis likely being preferred. Purity can be measured by any appropriate method, e.g., column chromatography, mass spectrometry, HPLC analysis, and the like.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

Phosphatidylinositol 4,5-bisphosphate (PIP₂) has the structure:

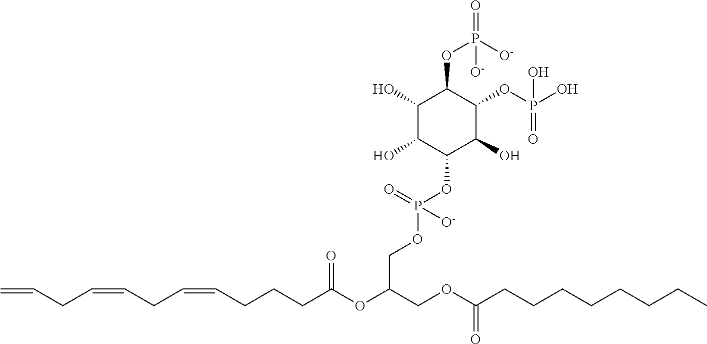

The activation of membrane receptors by hormones and growth factors results in the localized generation of intracellular second messengers. The hydrolysis of membrane phospholipids and the generation of biologically active products play important roles in the regulation of cell function and cell fate. Phosphoinositide-specific phospholipase C (PLC) isoforms hydrolyze phosphatidylinositol 4,5-bisphosphate (PIP₂), a membrane phospholipid found in all eukaryotic cells. PIP₂ is a critical cofactor for PLD, Phospholipase D (PLD), (which hydrolyzes phosphatidylcholine to yield phosphatidic acid (PA) and choline) and profoundly affects the activity, membrane localization and receptor activation of both PLD isoforms, PLD1 and PLD2. Thus, reduction of cellular PIP₂ levels has been shown to inhibit PLD activity. Vice versa, the synthesis of PIP₂ by phosphoinositide 5-kinase (PIP5K) isoforms can be directly stimulated by the PLD product PA. A review of the PIP-2 metabolic pathways is found in De Matteis et al. (2004) Nature Cell Biology 6:487, herein specifically incorporated by reference.

Molecules that inhibit the enzymatic pathways responsible for production of PIP-2 are of interest for use in the methods of the invention. Such inhibitors include, without limitation, inhibitors of phosphatidylinositol 4-kinase III alpha (see, for example Berger et al. (2009) PNAS 106:7577-7582, herein specifically incorporated by reference). Such enzymes may be inhibited, for example, with sequence specific inhibitors, such as specific antisense, RNAi, siRNA, etc. Alternatively, small molecule inhibitors may be used.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, 13-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

“Operably linked” refers to an arrangement of elements wherein the components so described are configured so as to perform their usual function. For example, a promoter that is operably linked to a coding sequence will effect the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

### Methods of the Invention

Contrary to the classic paradigm of anti-infective therapy, the present invention provides methods of treating viral infection by targeting a host function and/or molecule upon which the pathogen is dependent, thereby decreasing the ability of the pathogen to avoid the therapeutic agent by mutation. In addition, by utilizing a novel target, the methods of the invention allow combination therapies in which multiple targets are addressed, thereby increasing the ability to eliminate the infectious agent. The methods also provide a broad platform for antiviral therapies by targeting a host function.

In some embodiments, where the pathogen is HCV, useful compounds include those having a high first-pass effect and consequent low systemic bioavailability, which are targeted to the liver, and which are typically discarded in early drug development. In other embodiments for the treatment of HCV, the compound, or formulation, is modified for liver specific targeting.

The Examples provide a demonstration of the importance of PIP2 binding for mediating viral genome replication, which plays an essential role in a diverse group of important pathogens. In particular, BAAPP domains are identified in key proteins of Dengue and Japanese encephalitis viruses, as well as Plasmodium falciparum (see FIG. 8). Because direct PIP2 binding, for example by neomycin, or inhibition of an enzyme responsible for generating a particular phosphoinositide isoform, is surprisingly well tolerated by the host cell, this provides for a novel anti-infective therapy.

### Screening Methods

The present invention provides methods of identifying agents that interfere with the PIP-2:BAAPP domain binding interaction, and that are useful as an anti-viral agent. In some embodiments, the PIP-2 binding affinity of a BAAPP domain-containing peptides is determined. For example a quartz crystal microbalance with dissipation (QCM-D) assay may be utilized wherein lipid vesicles containing small amounts of PIP2 (or other mono-, di-, and tri-phosphoinositide lipids for controls) are deposited on an oscillating quartz crystal nanosensor and the change in resonant frequency upon introduction of wild-type or BAAPP domain mutant peptides in the flow chamber is directly proportional to the change in bound mass. This sensitive technique is ideal for determination of binding kinetics. CD measurements on the peptides assess the change in conformation observed upon PIP2 binding (see Examples).

For screening of candidate agents, a BAAPP domain PIP2 binding assay may be utilized. Peptides containing a BAAPP domain as described herein are adhered to a plate, e.g. a microtiter plate by any convenient method. Binding of PIP2-containing lipid vesicles is in the absence or presence of a candidate agent, preferably utilizing positive and negative controls. The lipid vesicles are conveniently labeled, e.g. with a fluorescent label, and monitored appropriately. Specificity controls may include BAAPP domain mutant peptide and lipid vesicles containing non-PIP2 phosphoinositide lipids. The assay may be validated using neomycin (a known PIP2 ligand) and various commercially-available structural analogues of neomycin. Such assays may be performed in a high throughput manner.

In some embodiments of the invention, detection of the PIP2:BAPP domain interaction is monitored by fluorescence polarization. Fluorescence polarization (FP) measurements are based on the assessment of the rotational motions of species. When linear polarized light is used to excite an ensemble of fluorophores, only those fluorophores aligned with the plane of polarization will be excited. If the fluorescence lifetime of the excited fluorescent probe is much longer than the rotational correlation time of the molecule it is bound to, the molecules will randomize in solution during the process of emission, and, as a result, the emitted light of the fluorescent probe will be depolarized. If the fluorescence lifetime of the fluorophore is much shorter than the rotational correlation time the excited molecules will stay aligned during the process of emission and as a result the emission will be polarized. Typically a sample containing a fluorescently labeled molecule is excited with linear polarized light and the vertical and horizontal components of the intensity of the emitted light are measured and the polarization or anisotropy are calculated. FP can be read on machines such as the AnalystGT, as known in the art. FP has an advantage that it requires only one labeled species for the assay, and thus FP is a particularly useful format for high throughput screening.

In FP screening of PIP2:BAPP domain interaction, PIP2 is preferably fluorescently labeled; and is brought into contact with a peptide comprising a suitable amphipathic helix, e.g. a polypeptide comprising the HCV NS5A BAPP domain amphipathic helix, where the polypeptide may comprise just the BAPP domain, may extend further into the NS5A protein, may be fused to a heterologous polypeptide, etc. Alternatively the polypeptide may be labeled. In other embodiments, a lipid other than PIP2 may be used. Other BAPP domain-containing polypeptides, as defined herein, may also be used. Generally such domains will retain PIP2 binding capability.

Fluorophores of interest include fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green); and the like. Fluorescein and rhodamine are of particular interest. Alternatively a class of dyes that have been shown to combine long lifetime and high polarization are the metal-ligand complexes of Ru, Os and Re.

FP assays are typically run with candidate inhibitors, e.g. in a series of dilutions, where the ability of the inhibitor to alter the interaction between PIP2 or an analogous lipid and a BAPP domain is read out by a change in fluorescence polarization. Controls include fluorescent versions of PI-lipids that are not PIP2, and mutant versions of the BAPP domain-containing peptide or protein wherein one of the amino acids of the “PIP2 pincer” is mutated so as to abrogate PIP2 binding.

Candidate agents that are positive in the assay may be further validated in models of viral replication, for example HCV replicon colony formation assays. Such assays monitor the replication efficiency of an HCV genome modified to encode resistance to a selectable marker such as blasticidin, and provide a quantitative measure of the frequency and strength of resistance emerging upon treatment with a given drug. This assay also allows one to readily determine whether phenotypically-resistant colonies are the result of specific adaptive mutations in the viral genome or host cell adaptations.

Compounds of interest for screening include analogs of PIP-2; neomycin and derivatives or analogs thereof, lithium, etc. Agents with high-first pass effect are of interest for hepatitis indications. Also of interest are liver targeted formulations via conjugation with bile acid (exploit entero-hepatic circulation). In other embodiments, drugs are provided in a prodrug form requiring hepatocyte activation of prodrug, for example cyp-mediated drug activation; HCV NS3 protease-mediated removal of prodrug-inactivating peptide; and the like. Compounds with good systemic bioavailability may be developed for the other pathogen indications.

Identification of anti-HCV agents according to the invention, and their use in inhibiting HCV replication and treating HCV infection, is of particular interest. The HCV contemplated by the invention may be of any genotype (genotype 1, 2, 3, 4, 5, 6, and the like), as well as subtypes of an HCV genotype (e.g., 1a, 1b, 2a, 2b, 3a, etc.)). Because currently HCV genotype 1 is normally the most difficult to treat, HCV genotype 1 and genotype 1 subtypes are of particular interest.

While the specification refers to HCV, such is only for clarity and is not intended to limit the invention. As noted above, the invention can be applied to a number of other BAAPP domain containing viruses.

BAAPP domain polypeptides that are suitable for use in a subject screening method include polypeptides that comprise BAAPP domains that bind (in a specific manner) PIP-2. Such domains may be a stretch of at least 10, at least 12, at least 14, at least 16, at least 20, at least 40, at least 45, at least 50, at least 75, at least 100, at least 125, at least 150, at least 175, at least 200 or more contiguous amino acids of a BAAPP containing protein. The peptide may be produced in any convenient manner, e.g. synthetically, by recombinant methods, and the like.

In some embodiments, a suitable BAAPP domain polypeptide comprises an amino acid sequence having at least about 75%, at least about 80%, at least about 85%, at least about 90%, at least about 95%, at least about 98%, at least about 99%, or 100%, amino acid sequence identity over a stretch of from about 10 contiguous amino acids to 20 contiguous amino acids of the amino acid sequences depicted in FIG. 8.

In some embodiments, a suitable BAAPP domain polypeptide is constructed from any alpha helical peptide, natural or synthetic, which is either intrinsically amphipathic or is made to be amphipathic through sequence alterations, which also has two or more positively charged residues, including but not limited to any combination of lysine, arginine, or histidine or other non-naturally occurring amino acid substitutes that are positively charged, such that the position of these positive charges help mediate PIP2 binding.

In some embodiments, an BAAPP domain polypeptide is a fusion protein, e.g., a polypeptide comprising a BAAPP domain and a heterologous polypeptide (e.g., a fusion partner), where suitable heterologous polypeptides (fusion partners) include, but are not limited to, an epitope tag (e.g., glutathione-S-transferase, hemagglutinin (HA; e.g., CYPYDVPDYA), FLAG (e.g., DYKDDDDK), c-myc (e.g., CEQKLISEEDL), and the like); a polypeptide that provides a detectable signal (e.g., an enzyme that converts a substrate into a product that can be detected colorimetrically, fluorimetrically, etc., where suitable enzymes include, but are not limited to luciferase, alkaline phosphatase, peroxidase, and the like; a fluorescent protein (e.g., a green fluorescent protein, a red fluorescent protein, a yellow fluorescent protein, etc.); a luminescent protein; etc.); a polypeptide that provides for ease of purification of the polypeptide (e.g., a metal ion affinity peptide e.g., (His)ₙ, e.g., 6His, and the like); glutathione-S-transferase; and the like); a polypeptide that provides for insertion into a eukaryotic cell membrane; a polypeptide that provides for solubility; a polypeptide that provides for attachment to another moiety, to a solid support, etc. The polypeptide can also be detectably labeled, e.g., with a radiolabel. In some embodiments, the polypeptide is biotinylated.

In some embodiments, the polypeptide or the PIP-2 that it used in the assay is detectably labeled, e.g., is directly detectably labeled. Suitable detectable labels include, e.g., radiolabels; enzymes that act on a substrate to yield a colored, luminescent, or fluorescent product; fluorescent proteins (a green fluorescent protein, a yellow fluorescent protein, a red fluorescent protein, etc.); a fluorophore (e.g., fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green); and the like. In some embodiments, the polypeptide, e.g. NS5A, NS4B, etc. is labeled during in vitro translation, e.g., using an in vitro transcription/translation system that includes a tRNA charged with a fluorescently labeled amino acid.

In some embodiments, the BAAPP domain polypeptide is immobilized on a solid support. The polypeptide can be immobilized on a solid support directly or indirectly. Indirect immobilization can be achieved by immobilizing onto a solid support an antibody, streptavidin, etc. that specifically binds the polypeptide.

An BAAPP domain polypeptide can be present in a subject assay method in an amount of from about 1 attomole to about 1 femtomole, from about 1 femtomole to about 1 picomole, from about 1 picomole to about 1 nanomole, from about 1 nanomole to about 50 nanomoles, from about 50 nanomoles to about 100 nanomoles, from about 100 nanomoles to about 500 nanomoles, from about 500 nanomoles to about 1 μmole, from about 1 μmole to about 50 μmoles, from about 50 μmoles to about 100 μmoles, from about 100 μmoles to about 500 μmoles, from about 500 μmoles to about 1 mmole, from about 1 mmole to about 50 mmole, from about 50 mmole to about 100 mmole, or greater than 100 mmole.

Test agents of interest decrease binding of a BAAPP domain polypeptide to the PIP-2 by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the binding in the absence of the test agent.

In some embodiments, a test agent that inhibits binding of a BAAPP domain polypeptide to the PIP-2 is further tested for its ability to inhibit HCV replication in a cell-based assay. In these embodiments, a test agent of interest is contacted with a mammalian cell that harbors all or part of an HCV genome; and the effect, if any, of the test agent on HCV replication is determined. Suitable cells include mammalian liver cells that are permissive for HCV replication, e.g., an immortalized human hepatocyte cell line that is permissive for HCV. For example, a suitable mammalian cell is Huh7 hepatocyte or a subclone of Huh7 hepatocyte, e.g., Huh-7.5. Suitable cell lines are described in, e.g., Blight et al. (2002) J. Virol. 76:13001; and Zhang et al. (2004) J. Virol. 78:1448. In some embodiments, the HCV genome in the cell comprises a reporter, e.g., a nucleotide sequence encoding luciferase, a fluorescent protein, or other protein that provides a detectable signal; and determining the effect, if any, of the test agent on HCV replication is achieved by detection of a signal from the reporter.

Thus, in some embodiments, a test agent of interest inhibits HCV replication by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the level of HCV replication in the absence of the test agent.

A variety of different test agents may be screened using a subject method. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, as is generally described in WO 94/243 14, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

As used herein, the term “determining” refers to both quantitative and qualitative determinations and as such, the term “determining” is used interchangeably herein with “assaying,” “measuring,” and the like.

In some embodiments, in addition to determining the effect of a test agent on inhibition of PIP-2 binding, test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2 H-tetrazolium bromide) assay, and the like. Agents that do not exhibit significant cytotoxic activity are considered candidate agents.

A variety of other reagents may be included in the screening assay. These include reagents like salts, neutral proteins, e.g. albumin, detergents, etc., including agents that are used to facilitate optimal binding activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity, but may also be optimized to facilitate rapid high-throughput screening. In some embodiments, between 0.1 hour and 1 hour, between 1 hour and 2 hours, or between 2 hours and 4 hours, will be sufficient.

Assays of the invention include controls, where suitable controls include a sample (e.g., a sample comprising the BAAPP domain polypeptide, and PIP-2, in the absence of the test agent). Generally a plurality of assay mixtures is run in parallel with different agent concentrations to obtain a differential response to the various concentrations. Typically, one of these concentrations serves as a negative control, i.e. at zero concentration or below the level of detection.

### Pharmaceutical Compositions

The above-discussed compositions can be formulated using well-known reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a PIP-2 ligand, analog, etc. is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, the PIP2 antagonist and an antiviral agent, e.g. interferon, ribavirin, Enfuvirtide; RFI-641 (4,4″-bis-{4,6-bis-[3-(bis-carbamoylmethyl-sulfamoyl)-phenylamino]-(1,3,5) triazin-2-ylamino}-biphenyl-2,2″-disulfonic acid); BMS-433771 (2H-Imidazo(4,5-c)pyridin-2-one, 1-cyclopropyl-1,3-dihydro-3-((1-(3-hydroxypropyl)-1H-benzimidazol-2-yl)methyl)); arildone; Pleconaril (3-(3,5-Dimethyl-4-(3-(3-methyl-5-isoxazolyl)propoxy)phenyl)-5-(trifluoromethyl)-1,2,4-oxadiazole); Amantadine (tricyclo[3.3.1.1.3,7]decane-1-amine hydrochloride); Rimantadine (alpha-methyltricyclo[3.3.1.1.3,7]decane-1-methanamine hydrochloride); Acyclovir (acycloguanosine); Valaciclovir; Penciclovir (9-(4-hydroxy-3-hydroxymethyl-but-1-yl)guanine); Famciclovir (diacetyl ester of 9-(4-hydroxy-3-hydroxymethyl-but-1-yl)-6-deoxyguanine); Gancyclovir (9-(1,3-dihydroxy-2-propoxymethyl)guanine); Ara-A (adenosine arabinoside); Zidovudine (3′-azido-2′,3′-dideoxythymidine); Cidofovir (1-[(S)-3-hydroxy-2-(phosphonomethoxy)propyl]cytosine dihydrate); Dideoxyinosine (2′,3′-dideoxyinosine); Zalcitabine (2′,3′-dideoxycytidine); Stavudine (2′,3′-didehydro-2′,3′-dideoxythymidine); Lamivudine ((−)-β-L-3′-thia-2′,3′-dideoxycytidine); Abacavir (1S,4R)-4-[2-amino-6-(cyclopropylamino)-9H-purin-9-yl]-2-cyclopentene-1-methanol succinate); Emtricitabine (−)-β-L-3′-thia-2′,3′-dideoxy-5-fluorocytidine); Tenofovir disoproxil (Fumarate salt of bis(isopropoxycarbonyloxymethyl) ester of (R)-9-(2-phosphonylmethoxypropyl)adenine); Bromovinyl deoxyuridine (Brivudin); Iodo-deoxyuridine (Idoxuridine); Trifluorothymidine (Trifluridine); Nevirapine (11-cyclopropyl-5,11-dihydro-4-methyl-6H-dipyrido[3,2-b:2′,3′-f][1,4]diazepin-6-one); Delavirdine (1-(5-methanesulfonamido-1H-indol-2-yl-carbonyl)-4-[3-(1-methylethyl-amino)pyridinyl)piperazine monomethane sulfonated); Efavirenz ((−)6-chloro-4-cyclopropylethynyl-4-trifluoromethyl-1,4-dihydro-2H-3,1-benzoxazin-2-one); Foscarnet (trisodium phosphonoformate); Ribavirin (1-β-D-ribofuranosyl-1H-1,2,4-triazole-3-carboxamide); Raltegravir (N-[(4-Fluorophenyl)methyl]-1,6-dihydro-5-hydroxy-1-methyl-2-[1-methyl-1-[[(5-methyl-1,3,4-oxadiazol-2-yl)carbonyl]amino]ethyl]-6-oxo-4-pyrimidinecarboxamide monopotassium salt); Neplanocin A; Fomivirsen; Saquinavir (SQ); Ritonavir ([5S-(5R,8R, 10R,11R)]-10-hydroxy-2-methyl-5-(1-methylethyl)-1-[2-(methylethyl)-4-thiazolyl]-3,6-dioxo-8,11-bis(phenylmethyl)-2,4,7,12-tetraazatridecan-13-oic acid 5-thiazolylmethyl ester); Indinavir ([(1S,2R,5(S)-2,3,5-trideoxy-N-(2,3-dihydro-2-hydroxy-1H-inden-1-yl)-5-[2-[[(1,1-dimethylethyl)amino]carbonyl]-4-pyridinylmethyl)-1-piperazinyl]-2-(phenylmethyl- -erythro)pentonamide); Amprenavir; Nelfinavir; Lopinavir; Atazanavir; Bevirimat; Indinavir; Relenza; Zanamivir; Oseltamivir; Tarvacin; etc. are administered to individuals in a formulation (e.g., in the same or in separate formulations) with a pharmaceutically acceptable excipient(s). The therapeutic PIP2 antagonist and second antiviral agent, as well as additional therapeutic agents as described herein for combination therapies, can be administered orally, subcutaneously, intramuscularly, parenterally, or other route. PIP2 antagonist and second antiviral agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The therapeutic agent(s) may administered in a unit dosage form and may be prepared by any methods well known in the art. Such methods include combining the compounds of the present invention with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant invention can be used. A wide variety of delivery devices and systems are known to those skilled in the art.

Although such may not be necessary, agents described herein can optionally be targeted to the liver, using any known targeting means. The inhibitors of the invention may be formulated with a wide variety of compounds that have been demonstrated to target compounds to hepatocytes. Such liver targeting compounds include, but are not limited to, asialoglycopeptides; basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates; lactose-PEG-poly-L-lysine conjugates; asialofetuin; and lactosylated albumin.

The terms “targeting to the liver” and “hepatocyte targeted” refer to targeting of an agent to a hepatocyte, particularly a virally infected hepatocyte, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the protease inhibitor agent administered to the subject enters the liver via the hepatic portal and becomes associated with (e.g., is taken up by) a hepatocyte.

HCV infection is associated with liver fibrosis and in certain embodiments the inhibitors may by useful in treating liver fibrosis (particularly preventing, slowing of progression, etc.). The methods involve administering an inhibitor of the invention as described above, in an amount effective to reduce viral load, thereby treating liver fibrosis in the subject. Treating liver fibrosis includes reducing the risk that liver fibrosis will occur; reducing a symptom associated with liver fibrosis; and increasing liver function.

Whether treatment with an agent as described herein is effective in reducing liver fibrosis is determined by any of a number of well-established techniques for measuring liver fibrosis and liver function. The benefit of anti-fibrotic therapy can be measured and assessed by using the Child-Pugh scoring system which comprises a multi-component point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

Treatment of liver fibrosis (e.g., reduction of liver fibrosis) can also be determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; Ill. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

In some embodiments, a therapeutically effective amount of an agent of the invention is an amount of agent that effects a change of one unit or more in the fibrosis stage based on pre- and post-therapy measures of liver function (e.g, as determined by biopsies). In particular embodiments, a therapeutically effective amount of an inhibitor reduces liver fibrosis by at least one unit in the Child-Pugh, METAVIR, the Knodell, the Scheuer, the Ludwig, or the Ishak scoring system.

Secondary, or indirect, indices of liver function can also be used to evaluate the efficacy of treatment. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score. An effective amount of an agent is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of an agent is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Qualitative or quantitative tests of functional liver reserve can also be used to assess the efficacy of treatment with an agent. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of an agent in this context can be regarded as an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with an agent is effective in reducing the incidence of a disorder associated with cirrhosis of the liver can readily be determined by those skilled in the art.

Reduction in HCV viral load, as well as reduction in liver fibrosis, can be associated with an increase in liver function. Thus, the invention provides methods for increasing liver function, generally involving administering a therapeutically effective amount of an agent of the invention. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of an agent in this context is one that is effective to increase liver function by at least about 10%, at least about 20%, at least about 30%, at least about 40%, at least about 50%, at least about 60%, at least about 70%, at least about 80%, or more. For example, a therapeutically effective amount of an agent is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of an agent is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

HCV infection is associated with hepatic cancer and in certain embodiments the present invention provides compositions and methods of reducing the risk that an individual will develop hepatic cancer. The methods involve administering an agent, as describe above, wherein viral load is reduced in the individual, and wherein the risk that the individual will develop hepatic cancer is reduced. An effective amount of an agent is one that reduces the risk of hepatic cancer by at least about 10%, at least about 20%, at least about 25%, at least about 30%, at least about 35%, at least about 40%, at least about 45%, at least about 50%, at least about 55%, at least about 60%, at least about 65%, at least about 70%, or more. Whether the risk of hepatic cancer is reduced can be determined in, e.g., study groups, where individuals treated according to the methods of the invention have reduced incidence of hepatic cancer.

### Subjects Amenable to Treatment Using the Agents of the Invention

Individuals who have been clinically diagnosed as infected with a virus, particularly HCV, are suitable for treatment with the methods of the present invention. Individuals who are infected with HCV are generally identified (diagnosed) as having HCV RNA in their blood, and/or having anti-HCV antibody in their serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1, or other HCV subtypes and quasispecies. Such individuals include naïve individuals (e.g., individuals not previously treated for HCV) and individuals who have failed prior treatment for HCV (“treatment failure” patients). Treatment failure patients include non-responders (e.g., individuals in whom the HCV titer was not significantly or sufficiently reduced by a previous antiviral treatment for HCV); and relapsers (e.g., individuals who were previously treated for HCV, whose HCV titer decreased, and subsequently increased). In particular embodiments of interest, individuals of interest for treatment according to the invention have detectable HCV titer indicating active viral replication, they may also have an HCV titer of at least about 10⁵, at least about 5×10⁵, or at least about 10⁶, or greater than 2 million genome copies of HCV per milliliter of serum.

### Determining Effectiveness of Antiviral Treatment

Whether a subject method is effective in treating a hepatitis virus infection, particularly an HCV infection, can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. For example, quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

As noted above, whether a subject method is effective in treating a hepatitis virus infection, e.g., an HCV infection, can be determined by measuring a parameter associated with hepatitis virus infection, such as liver fibrosis. Liver fibrosis reduction can be assessed by a variety of serum-based assay or by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As one non-limiting example, levels of serum alanine aminotransferase (ALT) are measured, using standard assays. In general, an ALT level of less than about 45 international units per milliliter serum is considered normal. In some embodiments, an effective amount of anti-HCV agent is an amount effective to reduce ALT levels to less than about 45 IU/m1 serum.

### BAAPP and Lipoproteins

In some embodiments of the invention, the screening methods, identification of BAAPP domains, etc are applied to lipoproteins, where agents that interfere with PIP-2 binding to lipoprotein BAAPP domains find use in the treatment of hyperlipdemia.

Hyperalphalipoproteinemia (HALP) is caused by a variety of genetic and environmental factors. Among these, plasma cholesteryl ester transfer protein (CETP) deficiency is the most important and frequent cause of HALP in the Asian populations. CETP facilitates the transfer of cholesteryl ester (CE) from high density lipoprotein (HDL) to apolipoprotein (apo) B-containing lipoproteins, and is a key protein in the reverse cholesterol transport system. The deficiency of CETP causes various abnormalities in the concentration, composition, and function of both HDL and low density lipoprotein (LDL).

Dyslipidemia is elevation of plasma cholesterol and/or TGs or a low HDL level that contributes to the development of atherosclerosis. Causes may be primary (genetic) or secondary. Diagnosis is by measuring plasma levels of total cholesterol, TGs, and individual lipoproteins. Phenotypes include the following:

Lipoprotein Patterns (Fredrickson Phenotypes)

| Phenotype | Elevated Lipoprotein(s) | Elevated Lipids |
|---|---|---|
| I | Chylomicrons | TGs |
| IIa | LDL | Cholesterol |
| IIb | LDL and VLDL | TGs and cholesterol |
| III | VLDL and chylomicron remnants | TGs and cholesterol |
| IV | VLDL | TGs |
| V | Chylomicrons and VLDL | TGs and cholesterol |

TGs = triglycerides;
LDL = low-density lipoprotein;
VLDL = very-low-density lipoprotein.

Primary causes are single or multiple genetic mutations that result in either overproduction or defective clearance of TG and LDL cholesterol, or in underproduction or excessive clearance of HDL. Primary lipid disorders are suspected when a patient has physical signs of dyslipidemia, onset of premature atherosclerotic disease (<60 yr), a family history of atherosclerotic disease, or serum cholesterol >240 mg/dL (>6.2 mmol/L). Primary disorders, the most common cause of dyslipidemia in children, do not cause a large percentage of cases in adults.

Secondary causes contribute to most cases of dyslipidemia in adults. The most important secondary cause in developed countries is a sedentary lifestyle with excessive dietary intake of saturated fat, cholesterol, and trans fatty acids (TFAs). TFAs are polyunsaturated fatty acids to which hydrogen atoms have been added; they are commonly used in many processed foods and are as atherogenic as saturated fat. Other common secondary causes include diabetes mellitus, alcohol overuse, chronic renal insufficiency and/or failure, hypothyroidism, primary biliary cirrhosis and other cholestatic liver diseases, and drugs, such as thiazides, β-blockers, retinoids, highly active antiretroviral agents, estrogen and progestins, and glucocorticoids.

Dyslipidemia itself causes no symptoms but can lead to vascular disease, including coronary artery disease and peripheral arterial disease. High TGs (>1000 mg/dL [>11.3 mmol/L]) can cause acute pancreatitis. High levels of LDL can cause eyelid xanthelasmas; arcus corneae; and tendinous xanthomas found at the Achilles, elbow, and knee tendons and over metacarpophalangeal joints. Patients with the homozygous form of familial hypercholesterolemia may have the above findings plus planar or cutaneous xanthomas. Patients with severe elevations of TGs can have eruptive xanthomas over the trunk, back, elbows, buttocks, knees, hands, and feet. Patients with the rare dysbetalipoproteinemia can have palmar and tuberous xanthomas.

Dyslipidemia is diagnosed by measuring serum lipids, though it may be suspected in patients with characteristic physical findings. Routine measurements (lipid profile) include total cholesterol (TC), TGs, HDL, and LDL.

Elevated LDLs: ATPIII guidelines recommend treatment for adults with elevated LDL levels and a history of CAD; conditions that confer a risk for future cardiac events similar to that of CAD itself (CAD equivalents, defined as diabetes mellitus, abdominal aortic aneurysm, peripheral arterial disease, and symptomatic carotid artery disease); or ≧2 CAD risk factors. ATPIII guidelines recommend that these patients have LDL levels lowered to <100 mg/dL, but accumulating evidence suggests that this target may be too high and a target LDL<70 mg/dL is an option for patients at very high risk (eg, those with known CAD and diabetes, other poorly controlled risk factors, metabolic syndrome, or acute coronary syndrome). When drugs are used, a dose providing at least a 30 to 40% decrease in LDL is desirable.

Procedural approaches are reserved for patients with severe hyperlipidemia (LDL>300 mg/dL) that is refractory to conventional therapy, such as occurs with familial hypercholesterolemia. Options include LDL apheresis (in which LDL is removed by extracorporeal plasma exchange), ileal bypass (to block reabsorption of bile acids), liver transplantation (which transplants LDL receptors), and portocaval shunting (which decreases LDL production by unknown mechanisms). LDL apheresis is the procedure of choice in most instances when maximally tolerated therapy fails to lower LDL adequately. Apheresis is also the usual therapy in patients with the homozygous form of familial hypercholesterolemia who have limited or no response to drug therapy. Because apoproteins are required for formation of LDL and VLDL, pharmacologic inhibition of BAAPP can modulate serum levels of LDL and VLDL.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### PIP2 is a Ligand of HCV NS5A and Mediates Viral Genome Replication

Phosphoinositides are important mediators of intracellular signaling and membrane trafficking pathways. Here we discovered a novel role for phosphatidylinositol-4,5-bisphosphate (PIP-2) as a key mediator of genome replication for hepatitis C virus (HCV), an important worldwide cause of liver disease. In particular, the N-terminal amphipathic helix (AH) of HCV nonstructural protein 5A (NS5A) was found to specifically bind lipid vesicles containing PIP-2. PIP-2 binding induced a significant conformational change in the AH. A pair of positively-charged lysine residues within the AH was found to be critical for mediating both PIP-2 binding and RNA genome replication. A similar structural motif is conserved across all HCV isolates as well as the AHs of many apolipoproteins. Finally, treatment with neomycin, a ligand of PIP-2, specifically inhibited HCV replication in a dose-dependent fashion. Together these results demonstrate the first example of phosphoinositides mediating viral genome replication, provide a molecular mechanism to explain the link between HCV and VLDL assembly, and suggest the potential for novel anti-HCV strategies.

Phosphoinositides (PIs) have long been known to mediate key intracellular signaling pathways. More recently, PIs have also been recognized as playing important roles in the subcellular localization of PI-interacting proteins which bind PIs via a variety of structural motifs. PIs such as phosphatidylinositol-4,5-bisphosphate (PIP-2) are recognized by, and can modulate the function of, several proteins involved in intracellular vesicular membrane trafficking, (amphiphysin). In the case of epsin, PIP-2 binding stabilizes an AH promoting membrane deformation important to membrane vesicle biogenesis . Hepatitis C virus (HCV) is an important of chronic liver disease. Current therapies are inadequate for many patients and new anti-HCV strategies are urgently needed. Because the HCV non-structural protein NS5A harbors an N-terminal AH essential for membrane-associated RNA replication, and NS5A has recently been found to interact with regulators of host cell vesicular membrane trafficking machinery, we hypothesized that the NS5A AH might also bind PIP-2 and that this interaction is essential for viral replication.

To test the hypothesis that the NS5A AH binds PIP2, we determined the ability of a synthetic peptide corresponding to the NS5A AH to bind to PIP2-containing lipid vesicles using a quartz crystal microbalance with dissipation (QCM-D) assay. In this assay, target lipid vesicles are coated on an oscillating quartz crystal nanosensor and the binding of peptide introduced into the flow chamber is directly proportional to the change in resonant frequency of the crystal upon peptide addition.

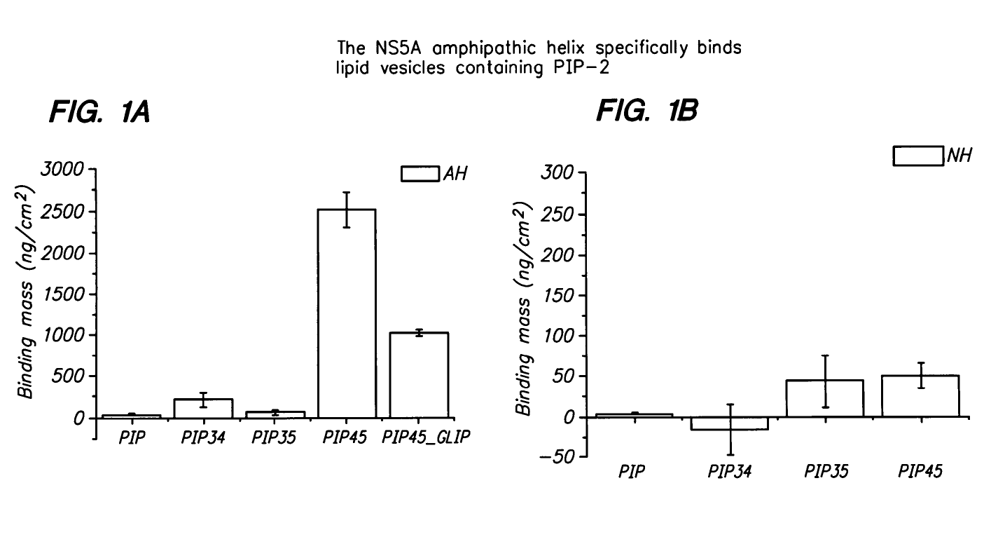

As shown in FIG. 1, significant binding of the NS5A AH to PIP2-containing vesicles was observed. The QCM-D technique allows for ready determination of binding kinetics. To determine the specificity of the observed binding, lipid vesicles containing phosphatidylinositol, phosphatidylinositol 3-4 phosphate, phosphatidylinositol 3-5 phosphate, or phosphatidylinositol 4-5 phosphate, were used in parallel assays. As shown in FIG. 1, there was a high degree of specificity observed for NS5A AH binding to PIP2. In particular, the related phosphatidylinositol bisbisphosphates, which have a similar number of negative charges to PIP2 had minimal binding to the NS5A AH.

Although a variety of structural motifs are known to bind PIP2, the NS5A AH does not appear to conform to any of these. As shown in FIG. 2, inspection of the NS5A AH revealed a pair of basic amino acids (Lys 20 and Lys 26) that flank the hydrophobic face of the AH. As such, they are oriented towards the lipid bilayer with which the AH likely interacts in a monotypic fashion. We hypothesized that these positively-charged lysines might be ideally-suited to interact with the negatively-charged phosphates of the PIP2 lipid headgroups. To test this hypothesis, we synthesized mutant versions of the NS5A AH peptide in which one or both of these lysines was mutated to alanine (FIG. 2). CD measurements confirmed that these mutations did not alter the helical nature of the AH. These mutations did, however, dramatically impair the ability of the corresponding peptides to bind PIP2. Taken together, these results demonstrate that the NS5A AH PIP2 binding domain represents a novel structural motif for PIP2 binding that we term Basic Amino Acid PIP2 Pincer or “BAAPP” domain.

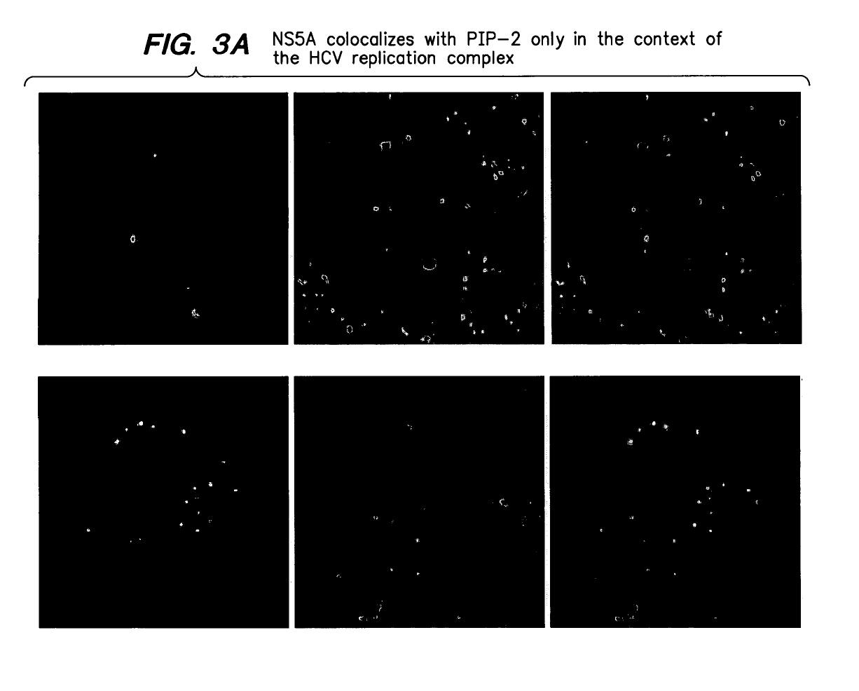

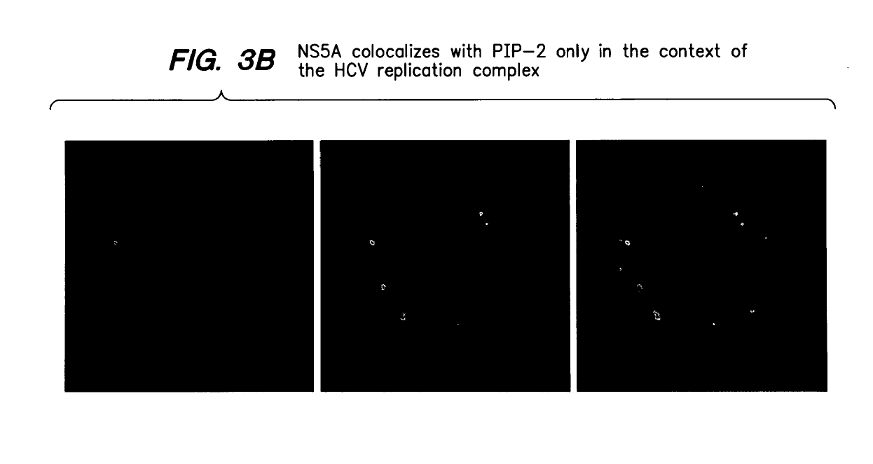

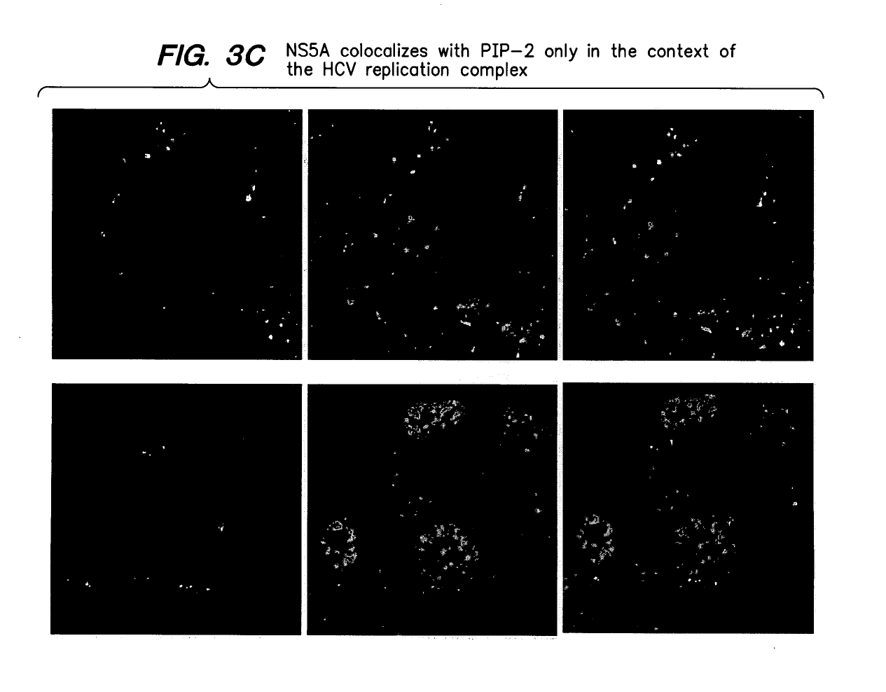

Because lysines 20 and 26 are highly conserved across HCV isolates in spite of sequence variation within the AH, this suggests that PIP2 binding by the NS5A AH is important for the HCV life cycle. We hypothesized that PIP2 binding might either mediate NS5A localization or an interaction important for replication. To test this hypothesis, we performed immunofluorescence colocalization studies of PIP2 with wild type (or mutant) versions of NS5A expressed either in isolation or in the context of an HCV replicon in Huh7 cells, a human liver tumor-derived cell line capable of supporting HCV genome replication. As shown in FIG. 3, a monoclonal antibody to PIP2 revealed both a nuclear staining pattern as well as distinct small speckles apparently distributed in the cytoplasm. Co-transfection of wild-type NS5A, or mutant NS5A (K2OAK26A—wherein lysines 20 and 26 are mutated to alanines), fused in frame to the N-terminus or either GFP or DsRed, respectively, yielded identical staining patterns on confocal microscopy for both versions of NS5A. Moreover, neither wild-type or mutant NS5A protein colocalized with PIP2. Similar results were obtained with unfused versions of wild type and mutant NS5A stained with an NS5A antibody, confirming that the ability to bind PIP2 had little apparent effect on the NS5A protein expressed in isolation.

Interestingly, however, in Huh7 cells harboring replicating HCV replicons significant colocalization of NS5A with PIP2 was observed. This was true for a replicons with a YFP-tagged version of NS5A or a wild-type replicon in which NS5A was detected by a specific antibody. Therefore, NS5A appears to co-localize with PIP2 only in the context of replication complex sites. This suggests that HCV replication complexes are established at PIP2 sites or the HCV replication complex promotes formation of PIP2 sites. Either way, PIP2 appears to represent a new marker for HCV replication complexes. These results also suggest that rather than representing an NS5A localization signal, PIP2 binding by NS5A might mediate an interaction important for replication. We hypothesized that this interaction involves a PIP2-induced conformational change in NS5A.

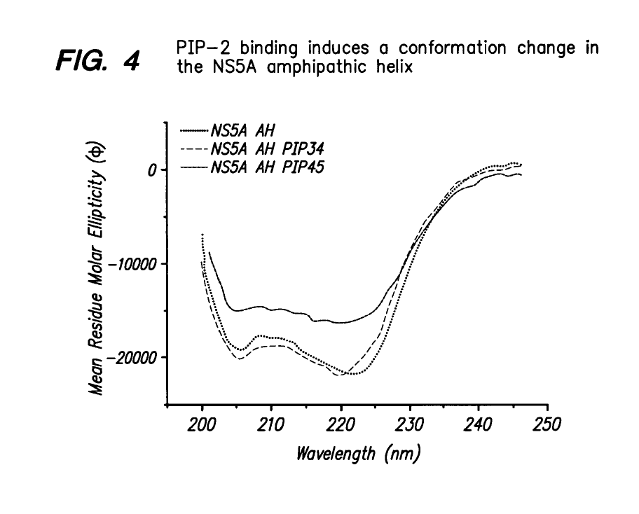

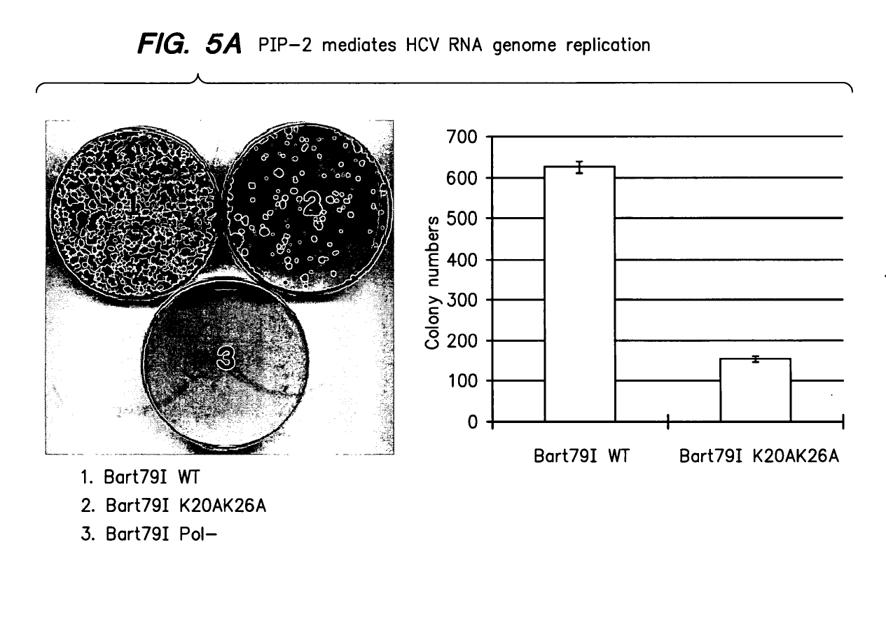

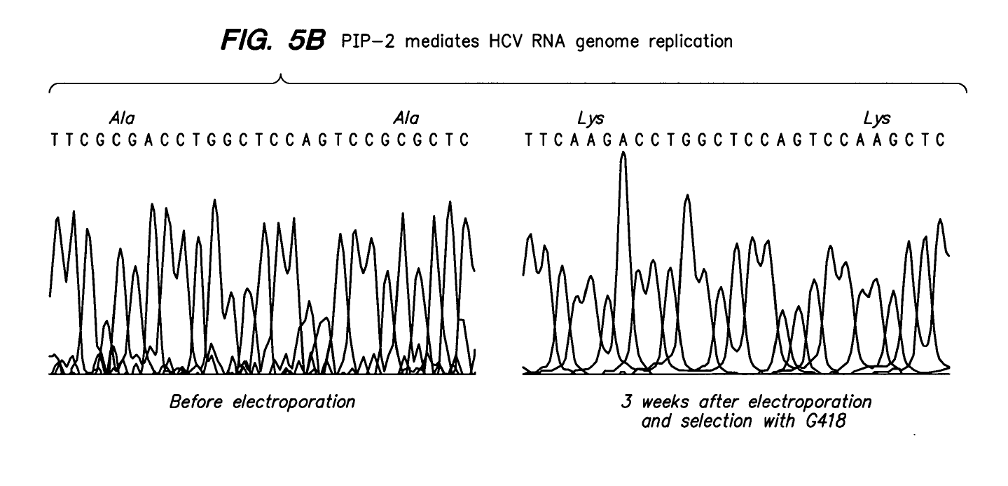

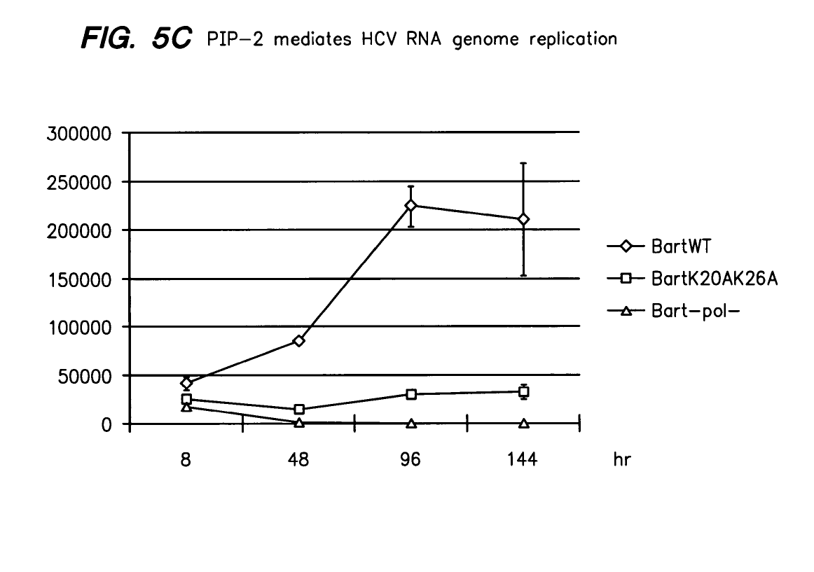

To test this hypothesis, CD measurements of the NS5A AH peptide were performed in the presence or absence of PIP2-containing lipid vesicles. As shown in FIG. 4, a dramatic alteration of the helical structure of the AH was observed upon interaction with PIP2. No such changes were noted with vesicles devoid of PIP2. Therefore PIP2 binding appears to mediate a conformational change in the AH of NS5A. To test the hypothesis that the ability to engage in this interaction is essential for replication, we first performed standard HCV colony formation assays using wild-type or NS5A mutant (K20AK26A) high efficiency second generation replicons. As shown in FIG. 5, while the wild-type replicon yielded numerous colonies and the negative control replicon containing a lethal mutation in the polymerase gene yielded none, ˜75% fewer colonies were obtained with the K20AK26A mutant compared to the wild-type. When the colonies growing on the mutant plate were examined in further detail, they were found to harbor replicons that had reverted to wild-type during the ˜3 week selection process, demonstrating that such reversion was essential for growth and that the ability of NS5A to bind PIP2 was important for efficient replication. To directly test this hypothesis, we performed transient HCV replication assays with luciferase reporter-linked wild-type or K20AK26A mutant replicons. As shown in FIG. 5, mutation of NS5A′s PIP2 interaction domain indeed severely impaired HCV genome replication. To our knowledge, this is the first example of PIP2 mediating viral genome replication.

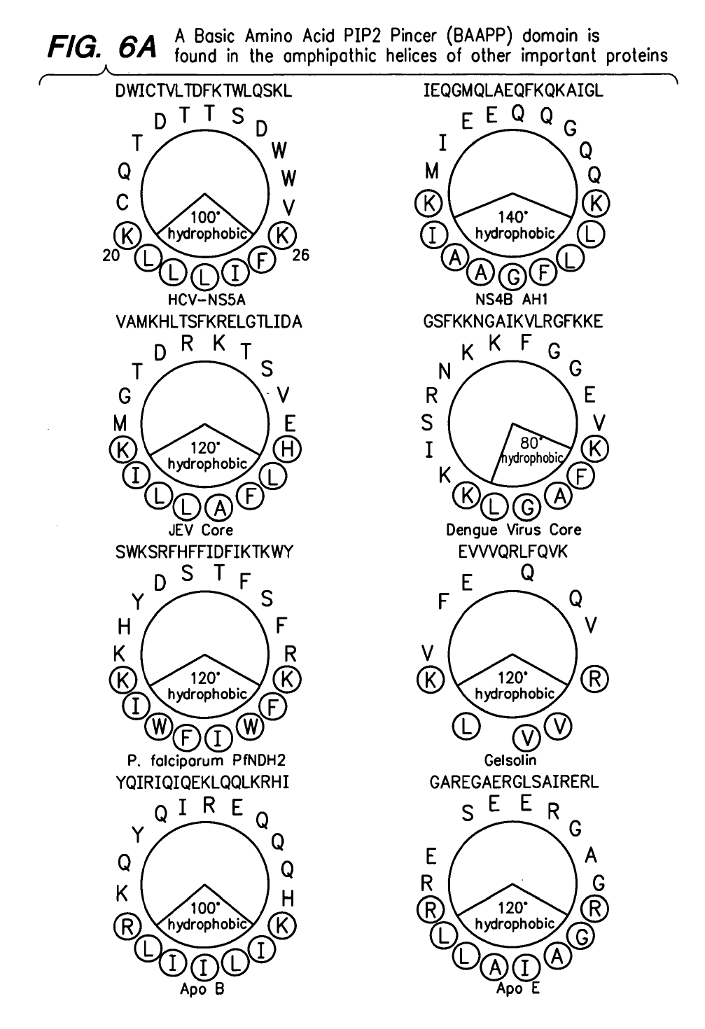

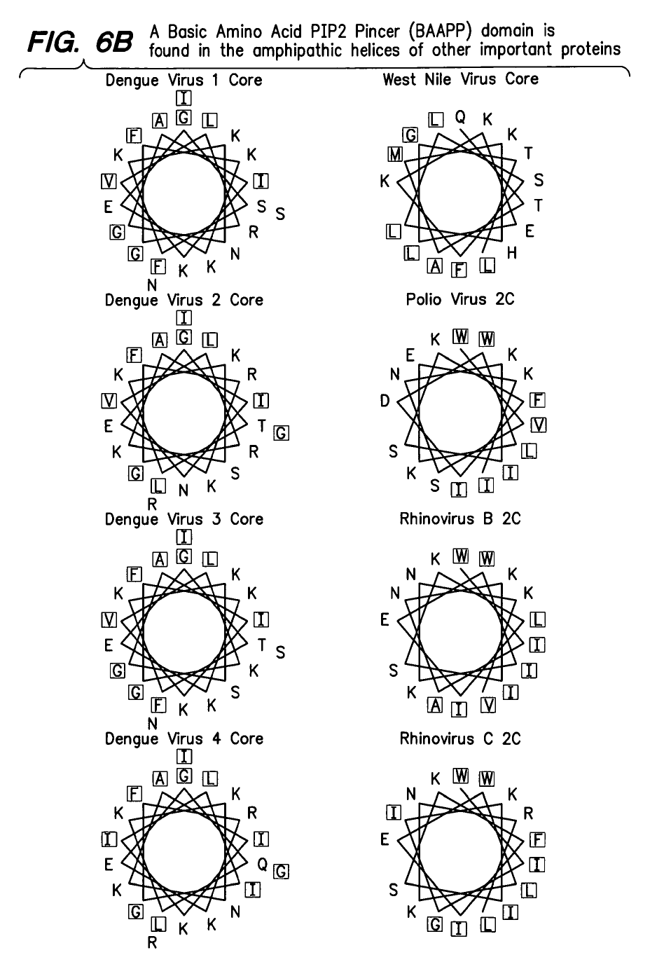

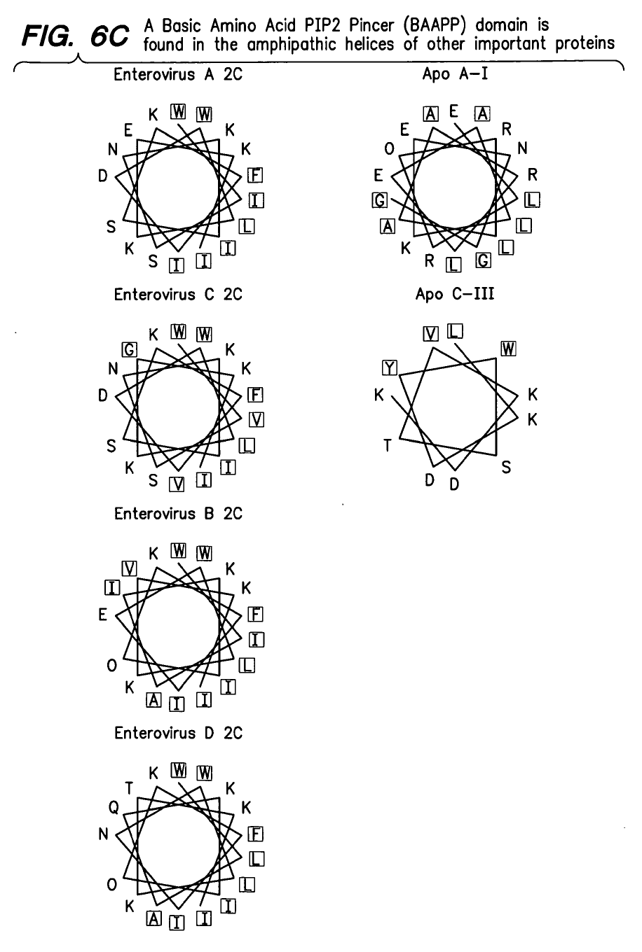

These results highlight the importance of the BAAPP domain in HCV-NS5A. Inspection of public databases reveals that BAAPP domains are present in a variety of other viral as well as host proteins. One important class of the latter is the apolipoproteins (see FIG. 6). These BAAPP domains may also mediate interaction with PIP2, providing an interaction that plays an important role in the genesis of certain lipoprotein particles. This provides a molecular mechanism to account for, among other things, the relationship between HCV and VLDL particle assembly. PIP2 domains may represent a common platform for the initial stages of VLDL lipoprotein and HCV particle assembly. In particular, HCV may either compete for or hijack limiting components of host cell PIP2-associated machinery to help effectuate viral assembly. This could account for the reciprocal relationship observed between serum levels of VLDL and HCV titer before and after successful treatment of HCV.

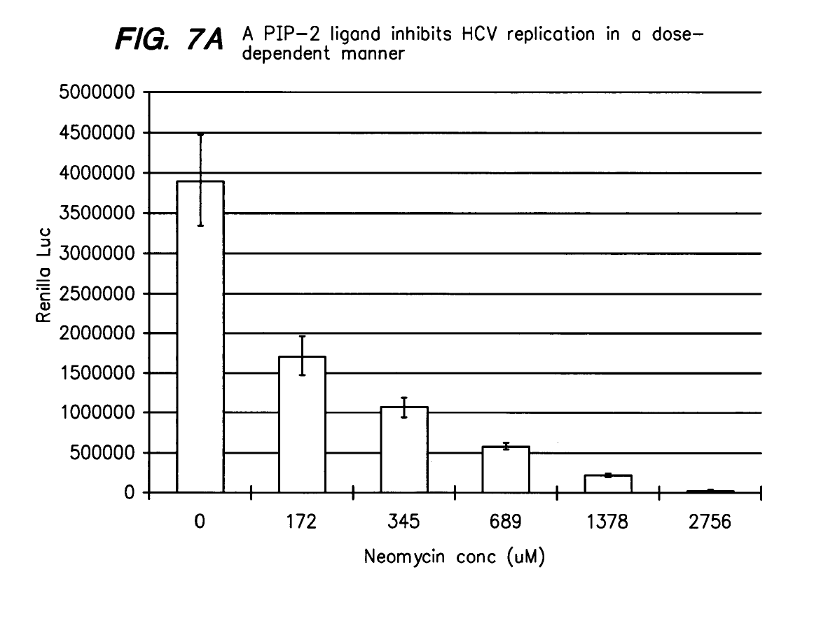

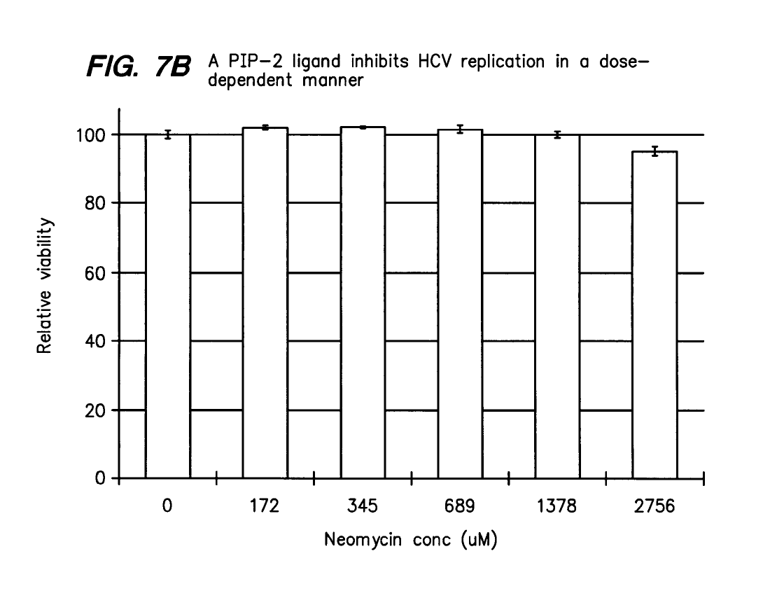

The NS5A PIP2 interaction is amenable to pharmacologic disruption. Transient HCV replication assays were performed with luciferase-linked replicons in the presence of increasing concentrations of neomycin, which is known to be a ligand of PIP2 and an inhibitor of PIP2 binding proteins. As shown in FIG. 7, a dose-dependent increase in inhibition of HCV genome replication was observed upon treatment with neomycin. No toxicity was observed until concentrations greater than 1 mM neomycin and an EC₅₀ of ˜200 μM was measured against HCV replication. It is not clear if sufficient anti-HCV hepatic concentrations are achieved with standard doses of neomycin, although a variety of known formulations might be exploitable to specifically increase hepatic concentrations of inhibitors of PIP2-BAAPP interactions. Such inhibitors represent a valuable new class of anti-HCV agents to be included in future therapeutic cocktails designed to maximize efficiency of, and minimize resistance to, therapies for treating hepatitis C.

### Materials and Methods

Plasmids. Bart79I, a high-efficiency subgenomic replicon of HCV, harbors the neomycin resistance gene (neo) and the HCV nonstructural proteins. Bart79-luc is constructed by replacing neo with firefly luciferase gene. The nucleotide sequence AAG that corresponds to lysine at the position of 20th and 26th amino acids of NS5A was changed to GCG (alanines) through the use of Quick-Change™XL site-directed mutagenesis kit (Stratagene, La Jolla, Calif.) as described by the manufacturer and confirmed by sequencing. For the neomycin treatment experiments, a modified Bart79I was used wherein the neo selection marker was replaced by the gene that confers resistance to blasticidin.

Immunofluorescence microscopy. Huh7 cells were grown on coverslips to 70% confluency. Coverslips were rinsed in phosphate-buffered saline (PBS) three times. They were fixed at room temperature for 15 min in 4% paraformaldehyde, permeabilized in 0.1% Triton-X in PBS for 5 min, rinsed three times in PBS, and blocked with PBS with 2% fetal bovine serum (FBS). Anti-PIP-2 (1:200, 2C11, Echelon Bioscience Inc., Salt Lake City, Utah) or anti-NS5A (1:1000, 6F3, Virostat, Portland, Me.) antibodies was applied, and the mixture was incubated for 2 hr. After three washes in PBS, coverslips were incubated with Alexa 594-conjugated anti-mouse IgM secondary antibody for PIP-2 or Alexa 488-conjugated anti-mouse IgG secondary antibody for NS5A (Invitrogen, Carlsbad, Calif.) for 1 hr. Following three washes with PBS, coverslips were mounted onto slides using Prolong Gold anti-fade reagent with DAPI (Invitrogen, Carlsbad, Calif.) and sealed. Fluorescent signals were examined and captured by Carl Zeiss confocal microscope.

Colony formation assay. 5 μg of in vitro-transcribed wild type and mutant Bart79I RNAs were mixed with 6×10⁶ cells in RNase-free PBS (Biowhittaker) and transferred into a 2 mm-diameter gap cuvette (BTX, San Diego, Calif.). Electroporation was performed using a BTX model 830 electroporator. The electroporation condition was as follows; 680 V, five periods of 99 μs at 500 ms intervals. The electroporated cells were diluted in 10 ml of cell culture medium. Cells were transferred to 10-cm tissue culture dishes at different dilutions. At 24 hr postelectroporation, cells were supplemented with untransfected feeder Huh-7 cells to a final density of 10⁶ cells/plate. Twenty-four hours later, the medium was supplemented with G418 to a final concentration of 750 μg/ml. This selection medium was replaced every 3 days for 3 weeks. Following selection, the plates were washed with PBS, incubated in 1% crystal violet in 20% ethanol for 5 min, and washed five times with H₂O for colony counting.

Viral sequencing analysis. Total RNA was isolated from the Huh7 cells electroporated with in vitro-transcribed wild type and mutant Bart79I RNAs with TRIzol reagent (Gibco BRL) as described by the manufacturer. SuperScript III one-step RT-PCR platinum Tag HiFi kit (Invitrogen, Carlsbad, Calif.) was used to revere transcribe HCV RNA into DNA first and then amplify NS5A region with two primers covering the entire NS5A sequence. Amplified PCR DNA fragments were purified by PCR purification kit from QIAGEN. Purified DNAs were sent to Sequetech Inc (Mountainview, Calif.) for sequencing analyses.

Transient replication assay. 10 μg of in vitro-transcribed wild type and mutant Bart79I-luciferase RNAs were electroporated into Huh7 cells as described above. The electroporated cells were diluted in 40 ml of cell culture medium. 2m1 of cells were aliquoted in 6 well tissue culture plates. Firefly luciferase activities were measured at 8, 48, 96, and 144 hr post electroporation by using firefly luciferase kit from Promega (Madison, Wis.).

For transient replication assay to study the effect of neomycin, 10 μg of in vitro-transcribed FL-J6/JFH-5′C19Rluc2AUbi RNAs were electroporated into Huh7 cells as described above. The electroporated cells were diluted in 18 ml of cell culture medium. 1 ml of cells was aliquoted in 6 well tissue culture plates. Electroporated cells were treated with 0, 172, 345, 689, 1378, and 2756 μM of neomyin for 5 days. Renilla luciferase activities were measured by using renilla luciferase kit from Promega (Madison, Wis.).

Cell viability assay. Cells were incubated with cell culture media containing 10% alamar blue (Biosource International, Inc., Camarillo, Calif.) for 2 hours. Relative cell viabilities were compared by measuring the absorbance of cell culture media at 544 nm.

Western blot analysis. Whole-cell extracts were prepared in RIPA buffer containing a cocktail of protease inhibitors (Complete, Mini; Roche Diagnostic) and quantitated by the Bradford assay (Bio-Rad). Equal amounts of protein were electrophoresed on an SDS-polyacrylamide gel, subsequently transferred to a polyvinylidene difluoride membrane (Immobilon-P; Millipore, Bedford, Mass.), and probed with anti-NS5A (1:500, 6F3, Virostat, Portland, Me.) antibody. Proteins were visualized via enhanced chemiluminescence (Amersham Pharmacia).

References include Balla 2005. J Cell Sci 118:2093-104; Blight et al. 2000. Science 290:1972-4; Brass et al. 2002. J Biol Chem 277:8130-9; Cantley 2002. Science 296:1655-7; Chauhan 1990 FEBS Lett 272:99-102; Cho et al. 2007J Virol 81:6682-9; De Matteis and Godi. 2004. Nat Cell Biol 6:487-92; Elazar, M., K. H. Cheong, P. Liu, H. B. Greenberg, C. M. Rice, and J. S. Glenn. 2003. Amphipathic helix-dependent localization of NS5A mediates hepatitis C virus RNA replication. J Virol 77:6055-61; Evans et al. 2004. Proc Natl Acad Sci USA 101:13038-43; Huang et al. 2007 Proc Natl Acad Sci USA 104:5848-53; Itoh et al. 2001 Science 291:1047-51; Nishizuka 1984 Science 225:1365-70; Ono et al. 2004 Proc Natl Acad Sci USA 101:14889-94; Osborne et al. 2001 J Cell Sci 114:2501-11; Schacht 1976 J Neurochem 27:1119-24; Schmidt, 2002 Nature 419:347-9; Sklan et al. 2007 J Virol 81:11096-105; Toker 2002. Cell Mol Life Sci 59:761-79; Tscherne et al. 2006 J Virol 80:1734-41.

1. A method of screening a candidate agent for anti-pathogen activity, the method comprising:
contacting a pathogen Basic Amino Acid PIP-2 Pincer (BAAPP) domain containing peptide with a phosphatidylinositol 4,5-bisphosphate (PIP-2) in the absence or presence of the candidate agent,
wherein an agent that specifically interferes with the interaction between the BAAPP domain and PIP-2 is a candidate for anti-pathogen activity.

2. The method of claim 1, further comprising determining the efficacy of the candidate agent in blocking pathogen replication.

3. The method of claim 1, wherein the BAAPP domain is derived from one of Rhinovirus B, Rhinovirus C, PolioVirus, Enterovirus A, Enterovirus B, Enterovirus C, Enterovirus D, Japanese Encephalitis Virus, West Nile Virus, Dengue Virus 1, Dengue Virus 2, Dengue Virus 3, Dengue Virus 4, P. falciparum, and hepatitis C virus.

4. The method of claim 3, wherein the virus is hepatitis C virus.

5. The method of claim 4, wherein the BAAPP domain is derived from NS5A protein.

6. The method of claim 4, wherein the BAAPP domain is derived from NS4B protein.

7. The method of claim 1, wherein ability of an agent to interfere with the interaction between the BAAPP domain and PIP-2 is determined by the method comprising:
contacting in a reaction a BAPP domain with fluorescently labeled PIP2 in the absence and presence of a candidate agent;
measuring fluorescence polarization of the reaction;
wherein an agent that interferes with the interaction will alter the fluorescence polarization.

8. A method of determining an interaction between a candidate BAAPP domain and PIP-2, the method comprising:
contacting a candidate peptide with lipid vesicles containing PIP-2, and determining the binding with a quartz crystal microbalance with dissipation (QCM-D) assay.

9. A method of inhibiting viral infection, the method comprising:
contacting virus-infected cells with an agent identified by the method set forth in claim 1 with a dose effective to inhibit viral replication.

10. The method of claim 9, further comprising administering a second antiviral agent.

11. The method of claim 9, wherein the agent is formulated to be targeted to the liver.

12. The method of claim 9, wherein the agent is neomycin or a derivative thereof.

13. The method of claim 9, wherein the agent is lithium or a derivative thereof.

14. A method of screening a candidate agent for activity in treating hyperlipidemia, the method comprising:
contacting a lipoprotein Basic Amino Acid PIP-2 Pincer (BAAPP) domain containing peptide with a phosphatidylinositol 4,5-bisphosphate (PIP-2) in the absence or presence of the candidate agent,
wherein an agent that specifically interferes with the interaction between the BAAPP domain and PIP-2 is a candidate for activity.
